SEQUENCE LISTING (30 sequences)

SEQ ID NO: 1 - protein, 241 aa, Artificial Sequence
  Synthetic Construct
EVQLQQSGAELVKPGASVKLSCTASGFNIKDTYIHWVKRRPEQGLEWIGRIDPANGNTKYDPKFQGKATITADTSSNTAYLQLSSLTSEDTAVYYCARRGLDYWGQGTLVTVSAGGGGSGGGGSGGGGSDVVMTQTPLTLSVTIGQPASISCKSSQSLLDGDGKTYLNWLLQRPGQSPKRLIYLVSKLDSGVPDRFTGSGSGTDFTLKISRVEAEDLGVYYCWQGTHFPRTFGGGTKLEIK

SEQ ID NO: 2 - DNA, 723 bp, Artificial Sequence
  Synthetic Construct
gaggtgcagctgcagcagagcggcgccgagctggtgaagcccggcgccagcgtgaagctgagctgcaccgccagcggcttcaacatcaaggacacctacatccactgggtgaagcgccgccccgagcagggcctggagtggatcggccgcatcgaccccgccaacggcaacaccaagtacgaccccaagttccagggcaaggccaccatcaccgccgacaccagcagcaacaccgcctacctgcagctgagcagcctgaccagcgaggacaccgccgtgtactactgcgcccgccgcggcctggactactggggccagggcaccctggtgaccgtgagcgccggtggaggcggttcaggcggaggtggctctggcggtggcggatcggacgtggtgatgacccagacccccctgaccctgagcgtgaccatcggccagcccgccagcatcagctgcaagagcagccagagcctgctggacggcgacggcaagacctacctgaactggctgctgcagcgccccggccagagccccaagcgcctgatctacctggtgagcaagctggacagcggcgtgcccgaccgcttcaccggcagcggcagcggcaccgacttcaccctgaagatcagccgcgtggaggccgaggacctgggcgtgtactactgctggcagggcacccacttcccccgcaccttcggcggcggcaccaagctggagatcaag

SEQ ID NO: 3 - protein, 241 aa, Artificial Sequence
  Synthetic Construct
EVQLQQSGAELVKPGASVKLSCTASGFNIKDTYIHWVKRRPEQGLEWIGRIDPANGNTKYDPKFQGKATITADTSSNTAYLQLSSLTSEDTAVYYCARRGGVDWGQGTLVTVSAGGGGSGGGGSGGGGSDVVMTQTPLTLSVTIGQPASISCKSSQSLLDGDGKTYLNWLLQRPGQSPKRLIYLVSKLDSGVPDRFTGSGSGTDFTLKISRVEAEDLGVYYCWQGTHFPRTFGGGTKLEIK

SEQ ID NO: 4 - DNA, 723 bp, Artificial Sequence
  Synthetic Construct
gaggtgcagctgcagcagagcggcgccgagctggtgaagcccggcgccagcgtgaagctgagctgcaccgccagcggcttcaacatcaaggacacctacatccactgggtgaagcgccgccccgagcagggcctggagtggatcggccgcatcgaccccgccaacggcaacaccaagtacgaccccaagttccagggcaaggccaccatcaccgccgacaccagcagcaacaccgcctacctgcagctgagcagcctgaccagcgaggacaccgccgtgtactactgcgcccgccgcggcggcgtggactggggccagggcaccctggtgaccgtgagcgccggtggaggcggttcaggcggaggtggctctggcggtggcggatcggacgtggtgatgacccagacccccctgaccctgagcgtgaccatcggccagcccgccagcatcagctgcaagagcagccagagcctgctggacggcgacggcaagacctacctgaactggctgctgcagcgccccggccagagccccaagcgcctgatctacctggtgagcaagctggacagcggcgtgcccgaccgcttcaccggcagcggcagcggcaccgacttcaccctgaagatcagccgcgtggaggccgaggacctgggcgtgtactactgctggcagggcacccacttcccccgcaccttcggcggcggcaccaagctggagatcaag

SEQ ID NO: 5 - protein, 241 aa, Artificial Sequence
  Synthetic Construct
QVQLVQSGAEVKKPGASVKVSCKASGFNIKDTYIHWVRQAPGQGLEWMGRIDPANGNTKYDPKFQGRVTMTRDTSISTAYMELSRLRSDDTAVYYCARRGGVDWGQGTLVTVSSGGGGSGGGGSGGGGSDIVMTQTPLSLSVTPGQPASISCKSSQSLLDGDGKTYLNWLLQKPGQSPQRLIYLVSKLDSGVPDRFSGSGSGTDFTLKISRVEAEDVGVYYCWQGTHFPRTFGQGTKLEIK

SEQ ID NO: 6 - DNA, 723 bp, Artificial Sequence
  Synthetic Construct
caggtgcagctggtgcagagcggcgccgaggtgaagaagcccggcgccagcgtgaaggtgagctgcaaggccagcggcttcaacatcaaggacacctacatccactgggtgcgccaggcccccggccagggcctggagtggatgggccgcatcgaccccgccaacggcaacaccaagtacgaccccaagttccagggccgcgtgaccatgacccgcgacaccagcatcagcaccgcctacatggagctgagccgcctgcgcagcgacgacaccgccgtgtactactgcgcccgccgcggcggcgtggactggggccagggcaccctggtgaccgtgagcagcggtggaggcggttcaggcggaggtggctctggcggtggcggatcggacatcgtgatgacccagacccccctgagcctgagcgtgacccccggccagcccgccagcatcagctgcaagagcagccagagcctgctggacggcgacggcaagacctacctgaactggctgctgcagaagcccggccagagcccccagcgcctgatctacctggtgagcaagctggacagcggcgtgcccgaccgcttcagcggcagcggcagcggcaccgacttcaccctgaagatcagccgcgtggaggccgaggacgtgggcgtgtactactgctggcagggcacccacttcccccgcaccttcggccagggcaccaagctggagatcaag

SEQ ID NO: 7 - protein, 236 aa, Artificial Sequence
  Synthetic Construct
EVQLQQSGAELVKPGASVKLSCTASGFDIKDTYIHWVKQRPEQGLEWIGRIDPANGDTRYDPEFQGKASLTADTSSNTAYLQFSNLTSEDTAVYYCARRGGLSWGQGTTLTVSSGGGGSGGGGSGGGGSDIQMTQSPASLSASVGETVTITCRASGNIHNYLAWYQQKQGKSPQLLVYNAKTLADGVPSRFSGSGSGTQYSLKINSLQPEDFGSYYCQHFWSTPWTFGGGTKLEIK

SEQ ID NO: 8 - DNA, 708 bp, Artificial Sequence
  Synthetic Construct
gaggttcaactgcaacagtctggggcagagcttgtgaagccaggggcctcagtcaagttgtcctgcacagcttctggcttcgacattaaagacacctatatccactgggtgaaacagaggcctgaacagggcctggagtggattggaaggattgatcctgcgaatggtgatactagatatgacccggaattccagggcaaggcctctctaacagctgacacatcctccaatacagcctacctacaattcagcaacctgacatctgaagacactgccgtctattactgtgctagaagaggcggcctctcctggggccaaggcaccactctcacagtctcctcaggcggcggcggcagcggcggcggcggcagcggcggcggcggcagcgacatccagatgactcagtctccagcctccctatctgcatctgtgggagaaactgtcaccatcacatgtcgagcaagtgggaatattcacaattatttagcatggtatcagcagaaacagggaaaatctcctcagctcctggtctataatgcaaaaaccttagcagatggtgtgccatcaaggttcagtggcagtggatcaggaacacaatattctctcaagatcaacagcctgcagcctgaagattttgggagttattactgtcaacatttttggagtactccgtggacgttcggtggaggcaccaagctggaaatcaaa

SEQ ID NO: 9 - protein, 249 aa, Artificial Sequence
  Synthetic Construct
QVQLKESGAELARPGASVKLSCKASGYTFTTFWMQWAKQRPGQGLEWIGTIYPGDDDTRYTQKFKGRATLTADKSSTTAYMQLSNLSSEDSAVYYCARSGYERGPYYFDSWGQGTTLTVSSGGGGSGGGGSGGGGSDTVMSQSPSSLAVSVGEKITMSCKSSQSLLCRSNQKNYLAWYQQKPGQSPKQLIYWASTRESGVPDRFTGSGSGTDFTLTISSVKAEDLAVYYCQQYYSYPLTFGAGTKLELK

SEQ ID NO: 10 - DNA, 747 bp, Artificial Sequence
  Synthetic Construct
caggtgcagctgaaggagtctggggctgagctggcaagacctggggcttcagtgaagttgtcctgcaaggcttctggctacacgtttactactttctggatgcagtgggcaaaacagaggcctggacagggtctggaatggattgggactatttatcctggagatgatgatactaggtacactcagaaattcaagggcagggccacattgactgcagataaatcgtccaccacagcctacatgcaactcagcaacttgtcatctgaggactctgcggtctattactgtgcaagatcggggtatgagaggggcccatactactttgactcctggggccaaggcaccactctcacagtctcctcaggcggcggcggcagcggcggcggcggcagcggcggcggcggcagcgacactgtgatgtcacagtccccatcctccctagctgtttcagttggagagaagataactatgagctgcaagtccagtcagagtcttttatgtcgtagcaatcaaaagaactacttggcctggtaccagcagaaaccagggcagtctcctaaacagctgatttactgggcatctactagggaatctggggtccctgatcgcttcacaggcagtggatctgggacagatttcactctcaccatcagcagtgtgaaggctgaagacctggcagtttattactgtcagcaatattatagttatccgctcacgttcggtgctggcaccaagctggagctgaaa

SEQ ID NO: 11 - protein, 21 aa, Artificial Sequence
  Synthetic Construct
MALPVTALLLPLALLLHAARP

SEQ ID NO: 12 - DNA, 63 bp, Artificial Sequence
  Synthetic Construct
atggccctgcctgtgacagctctgctcctccctctggccctgctgctccatgccgccagaccc

SEQ ID NO: 13 - protein, 14 aa, Artificial Sequence
  Synthetic Construct
NWSHPQFEKGGGGS

SEQ ID NO: 14 - DNA, 42 bp, Artificial Sequence
  Synthetic Construct
aactggagccacccccagttcgagaagggcggtggcggaagc

SEQ ID NO: 15 - protein, 45 aa, Artificial Sequence
  Synthetic Construct
TTTPAPRPPTPAPTIASQPLSLRPEACRPAAGGAVHTRGLDFACD

SEQ ID NO: 16 - DNA, 135 bp, Artificial Sequence
  Synthetic Construct
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 17 - protein, 27 aa, Artificial Sequence
  Synthetic Construct
FWVLVVVGGVLACYSLLVTVAFIIFWV

SEQ ID NO: 18 - DNA, 81 bp, Artificial Sequence
  Synthetic Construct
ttttgggtgctggtggtggttggtggagtcctggcttgctatagcttgctagtaacagtggcctttattattttctgggtg

SEQ ID NO: 19 - protein, 41 aa, Artificial Sequence
  Synthetic Construct
RSKRSRLLHSDYMNMTPRRPGPTRKHYQPYAPPRDFAAYRS

SEQ ID NO: 20 - DNA, 123 bp, Artificial Sequence
  Synthetic Construct
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 21 - protein, 42 aa, Artificial Sequence
  Synthetic Construct
KRGRKKLLYIFKQPFMRPVQTTQEEDGCSCRFPEEEEGGCEL

SEQ ID NO: 22 - DNA, 126 bp, Artificial Sequence
  Synthetic Construct
aaacggggcagaaagaaactcctgtatatattcaaacaaccatttatgagaccagtacaaactactcaagaggaagatggctgtagctgccgatttccagaagaagaagaaggaggatgtgaactg

SEQ ID NO: 23 - protein, 112 aa, Artificial Sequence
  Synthetic Construct
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 24 - DNA, 336 bp, Artificial Sequence
  Synthetic Construct
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 25 - protein, 25 aa, Artificial Sequence
  Synthetic Construct
GSGVKQTLNFDLLKLAGDVESNPGP

SEQ ID NO: 26 - DNA, 75 bp, Artificial Sequence
  Synthetic Construct
ggaagcggagtgaaacagactttgaattttgaccttctcaagttggcgggagacgtggagtccaaccctggacca

SEQ ID NO: 27 - protein, 177 aa, Artificial Sequence
  Synthetic Construct
MFHVSFRYIFGLPPLILVLLPVASSDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 28 - DNA, 531 bp, Artificial Sequence
  Synthetic Construct
atgttccatgtttcttttaggtatatctttggacttcctcccctgatccttgttctgttgccagtagcatcatctgattgtgatattgaaggtaaagatggcaaacaatatgagagtgttctaatggtcagcatcgatcaattattggacagcatgaaagaaattggtagcaattgcctgaataatgaatttaacttttttaaaagacatatctgtgatgctaataaggaaggtatgtttttattccgtgctgctcgcaagttgaggcaatttcttaaaatgaatagcactggtgattttgatctccacttattaaaagtttcagaaggcacaacaatactgttgaactgcactggccaggttaaaggaagaaaaccagctgccctgggtgaagcccaaccaacaaagagtttggaagaaaataaatctttaaaggaacagaaaaaactgaatgacttgtgtttcctaaagagactattacaagagataaaaacttgttggaataaaattttgatgggcactaaagaacac

SEQ ID NO: 29 - protein, 98 aa, Artificial Sequence
  Synthetic Construct
MALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 30 - DNA, 294 bp, Artificial Sequence
  Synthetic Construct
atggccctgctactggccctcagcctgctggttctctggacttccccagccccaactctgagtggcaccaatgatgctgaagactgctgcctgtctgtgacccagaaacccatccctgggtacatcgtgaggaacttccactaccttctcatcaaggatggctgcagggtgcctgctgtagtgttcaccacactgaggggccgccagctctgtgcacccccagaccagccctgggtagaacgcatcatccagagactgcagaggacctcagccaagatgaagcgccgcagcagt

(19) United States
(12) Patent Application Publication
(10) Pub. No.: US 2024/0317882 A1
(43) Pub. Date: Sep. 26, 2024

(54) CD99-targeting chimeric antigen receptor and application thereof

(71) Applicant: WUHAN BIO-RAID BIOTECH CO., LTD, Wuhan, Hubei (CN)

(72) Inventors: Tongcun ZHANG, Wuhan, Hubei (CN); Haichuan ZHU, Wuhan, Hubei (CN); Jiangzhou SHI, Wuhan, Hubei (CN)

(21) Appl. No.: 18/557,875

(22) PCT Filed: Nov. 19, 2021
(86) PCT No.: PCT/CN2021/131612

§ 371 Date: Oct. 27, 2023

(30) Foreign Application Priority Data
Apr. 30, 2021 (CN) 202110485551.1
May 8, 2021 (CN) 202110501688.1

(51) Int. Cl. C07K 16/28; A61K 39/00; A61P 35/00; A61P 35/02; C07K 14/54; C07K 14/705; C07K 14/725; C12N 15/86

(52) U.S. Cl. CPC C07K 16/2896; A61K 39/4611; A61K 39/4631; A61K 39/4635; A61K 39/464429; A61P 35/00; A61P 35/02; C07K 14/5418; C07K 14/7051; C07K 14/70517; C07K 14/70521; C07K 14/70578; C12N 15/86; A61K 2239/15; A61K 2239/17; A61K 2239/21; A61K 2239/22; C07K 2317/53; C07K 2317/622; C07K 2319/03; C07K 2319/22; C12N 2740/15043

(57) ABSTRACT

Disclosed herein is an optimized CD99-targeting chimeric antigen receptor and application thereof, wherein a signal peptide, a single-chain antibody ScFv, strepII, a CD8 hinge, a CD28 transmembrane region, a CD28 intracellular domain, an intracellular co-stimulatory domain 4-1BB and CD3ζ chain are sequentially spliced in the chimeric antigen receptor is from the N-terminal to the C-terminal. The single-chain antibody ScFv can specifically recognize CD99 protein on the surface of tumor cells. The CD99-targeting chimeric antigen receptor is used to modify immune cells for the treatment of surface CD99-positive tumors.

### CROSS-REFERENCE TO RELATED APPLICATIONS

This application is a national stage application of PCT/CN2021/131612. This application claims the benefit of PCT Application No. PCT/CN2021/131612, filed Nov. 19, 2021, Chinese Application No. 2021104855511 filed on Apr. 30, 2021, and Chinese Application No. 2021105016881 filed on May 8, 2021. The content of which are incorporated herein in the entirety by reference.

### INCORPORATION-BY-REFERENCE OF SEQUENCE LISTING OR TABLE

The material in the accompanying sequence listing is hereby incorporated by reference in its entirety into this application. The accompanying file, named 43425_SubstituteSequenceListing.txt was created on Jun. 11, 2024 and is 27 KB.

### TECHNICAL FIELD

The present disclosure relates to the field of medical biology, in particular to a CD99-targeting chimeric antigen receptor (CAR) for treating a broad-spectrum of tumors and application thereof.

### BACKGROUND

Although there are many successes in the field of CAR-T cell therapy for cancers, some problems still exist in the process of such a therapy, including off-target caused by high heterogeneity of tumors and treatment problems arose from a single target, etc. Among others, the high heterogeneity of tumor cells directly leads to the limitation of CAR-T therapy in the course of treatment, while the single target limits the broad spectrum of treatment. Therefore, the selection of membrane surface markers with specific expression in tumor cells and a broad-spectrum of expression in different types of tumors is a crucial step for the effectiveness of CAR-T therapy, and it is still a huge challenge to obtain drugs with stronger targeted killing effect on tumors.

### SUMMARY

According to various embodiments of the present application, an optimized ScFv sequence and a CAR structure carrying the sequence are particularly provided. In some embodiments, the CAR structures provided exhibit significantly enhanced tumor killing effect compared to one or more reference CARs, and more particularly, in some embodiments, the CARs provided are effective in killing any tumor cells that express CD99 on their surface.

Specifically, according to the first aspect of the present application, a chimeric antigen receptor CAR is provided, in which a signal peptide, a single-chain antibody ScFv, strepII, a CD8 hinge, a CD28 transmembrane region, a CD28 intracellular domain, an intracellular co-stimulatory domain 4-1BB and CD3ζ chain are sequentially spliced from the N terminal to the C terminal; and preferably, F2A peptide, IL-7, F2A peptide and CCL19 are further spliced at the C terminal of CD3ζ chain; and the single-chain antibody ScFv is capable of identifying CD99 antigen on the surface of tumor cells. The nucleotide sequence of the signal peptide is shown as SEQ ID NO.12, the nucleotide sequence of strepII is shown as SEQ ID NO.14, the nucleotide sequence of CD8 hinge is shown as SEQ ID NO.16, The nucleotide sequences of the CD28 transmembrane region and the CD28 intracellular domain are shown as SEQ ID NO.18 and SEQ ID NO.20, respectively, the nucleotide sequence of the intracellular co-stimulatory domain 4-1BB is shown as SEQ ID NO.22, and the nucleotide sequence of CD3ζ is shown as SEQ ID NO.24.

In some embodiments of the present disclosure, the amino acid sequence of the single-chain antibody ScFv is shown as SEQ ID NO.1, and preferably, the nucleotide sequence of the single-chain antibody ScFv is shown as SEQ ID NO.2.

In some embodiments of the present disclosure, a signal peptide, a single-chain antibody ScFv, strepII, a CD8 hinge, a CD28 transmembrane region, a CD28 intracellular domain, an intracellular co-stimulatory domain 4-1BB, CD3ζ chain, F2A peptide, IL-7, F2A peptide, and CCL19 are sequentially spliced from the N-terminal to the C-terminal, and preferably, the amino acid sequence of the F2A peptide is shown as SEQ ID NO.25, the amino acid sequence of IL-7 is shown as SEQ ID NO.27, and the amino acid sequence of CCL19 is shown as SEQ ID NO.29, and more preferably, the nucleotide sequence of the F2A peptide is shown as SEQ ID NO.26, the nucleotide sequence of IL-7 is shown as SEQ ID NO.28, and the nucleotide sequence of CCL19 is shown as SEQ ID NO.30. Preferably, the amino acid sequence of the single-chain antibody ScFv is represented by SEQ ID NO.1, SEQ ID NO.3 or SEQ ID NO.5, and the nucleotide sequence of the single-chain antibody ScFv is represented by SEQ ID NO.2, SEQ ID NO.4 or SEQ ID NO.6; and most preferably, the amino acid sequence of the single-chain antibody ScFv is shown as SEQ ID NO.5, and the nucleotide sequence of the single-chain antibody ScFv is shown as SEQ ID NO.6.

According to the second aspect of the present disclosure, provided is a recombinant vector of the chimeric antigen receptor gene, which is a lentivirus, adenovirus, adeno-associated virus, retrovirus, or transposon vector in which a nucleotide sequence encoding the chimeric antigen receptor is inserted, using a viral or non-viral expression vector as a backbone. Preferably, the vector is a lentiviral vector using the virus vector PTK881-EF1α as a backbone, and with the above nucleotide sequence encoding the chimeric antigen receptor inserted; and the virus vector PTK881-EF1α is a vector obtained by replacing a CMV promoter with an EF1α promoter using a PTK881 vector as a backbone.

According to the third aspect of the present disclosure, there is provided an immune cell of the chimeric antigen receptor obtained by transfecting an immune cell with the nucleotide sequence encoding the chimeric antigen receptor or the recombinant vector of the chimeric antigen receptor gene.

In some embodiments of the present disclosure, the immune cell is selected from the group consisting of T-cell, NK cell, NKT cell, αβT cell, γδT cell, CD4+ T-cell, CD8+ T-cell derived from umbilical cord blood, peripheral blood or IPSC, and preferably T-cell derived from peripheral blood, that is, the CD99-targeting CAR-T cell for the treatment of a broad spectrum of tumors. When the single-chain antibody ScFv of the chimeric antigen receptor CAR binds to CD99, the immune cell expressing the chimeric antigen receptor exhibits anti-tumor activity. Preferably, the immune cell can be modified by CRISPR, RNA interference and other technologies in combination with the expression of chimeric antigen receptor elements.

According to the fourth aspect of the present disclosure, there is provided the use of the above nucleotide sequence encoding the chimeric antigen receptor, the above recombinant vector of the chimeric antigen receptor gene, and the above chimeric antigen receptor-expressing immune cell (CAR-T cell), including the preparation of drugs or kits for the treatment, prevention, and diagnosis of tumors. The tumor is preferably Ewing's sarcoma, acute lymphoma/leukemia, acute myeloid leukemia, malignant glioma, breast cancer, and more preferably, acute T-cell lymphoid leukemia.

In some embodiments of the present disclosure, the cell line selected for in vitro functional assay of the immune cell expressing the chimeric antigen receptor is a cell line with the CD99 target highly or moderately expressed outside the cell membrane.

According to the fifth aspect of the present disclosure, there is provided a method for preparing the immune cell expressing the chimeric antigen receptor, which comprises activating the isolated immune cell for 2-15 days, and then infecting the activated immune cell with the lentivirus expressing the chimeric antigen receptor.

### BRIEF DESCRIPTION OF THE DRAWINGS

FIG. 1 is a schematic diagram showing the DNA fragments of C6-CAR, C2-CAR, C3-CAR, C4-CAR, and C5-CAR in Examples.

FIG. 2 shows the DNA fragments of C6-7x19 CAR, C2-7x19 CAR, C3-7x19 CAR, C4-7x19 CAR, and C5-7x19 CAR in the Examples.

FIG. 3 is a plasmid map of PTK881-EF1α-C6, PTK881-EF1α-C2, PTK881-EF1α-C3, PTK881-EF1α-C4, and PTK881-EF1α-C5 in the Examples.

FIG. 4 is a plasmid map of PTK881-EF1α-C6-7x19, PTK881-EF1α-C2-7x19, PTK881-EF1α-C3-7x19, PTK881-EF1α-C4-7x19, and PTK881-EF1α-C5-7x19 in the Examples.

FIG. 5 is a schematic diagram showing the results of cell transduction efficiency after transfection by C6-CAR-T at different time points.

FIG. 6 is a schematic diagram showing the results of cell viability after transfection by C6-CAR-T at different time points.

FIG. 7 is a schematic diagram showing the in vitro killing effect of the CAR-T cells on Ewing's sarcoma cell lines TC71 and 6647.

FIG. 8 is a schematic diagram showing the in vitro killing results of the CAR-T cells on acute lymphoblastic lymphoma cell lines JURKAT and MOLT-4.

FIG. 9 shows the in vitro killing and lysis effect of the CAR-T cells on acute myeloid leukemia cell lines and breast cancer cell lines MOLM-13 and MCF-7.

FIG. 10 is a schematic diagram showing the in vitro killing results of the CAR-T cells on malignant glioma cell lines U373-MG and U251-MG.

FIG. 11 is a schematic diagram showing the in vitro killing results of the CAR-T cells on the negative cell line Raji.

FIG. 12 is a schematic diagram showing the results of cytokine IFN-γ release after in vitro co-incubation of the CAR-T cells with Ewing's sarcoma cell lines TC71 and 6647.

FIG. 13 is a schematic diagram showing the results of cytokine IFN-γ release after in vitro co-incubation of the CAR-T cells with acute lymphoblastic lymphoma cell lines JURKAT and MOLT-4.

FIG. 14 is a schematic diagram showing the results of cytokine IFN-γ release after in vitro co-incubation of the CAR-T cells with acute myeloid leukemia cell lines and breast cancer cell lines MOLM-13 and MCF-7.

FIG. 15 is a schematic diagram showing the results of cytokine IFN-γ release after in vitro co-incubation of the CAR-T cells with malignant glioma cell lines U373-MG and U251-MG.

FIG. 16 is a schematic diagram showing the results of cytokine IFN-γ release after in vitro co-incubation of the CAR-T cells with the negative cell line Raji.

### EXAMPLE

The scheme of the present disclosure will be explained below with reference to the Examples. Those skilled in the art will appreciate that the following Examples are merely illustrative of the present disclosure and should not be construed as limiting the scope of the present disclosure. If no specific technique or condition is indicated in the Examples, the technique or condition described in the publications in the field or in the product specification shall be followed. If the manufacturer of the reagent or instrument used is not specified, it is a conventional product that can be commercially available.

### Example 1: Affinity Assay of Anti-CD99 scFv

After several rounds of screening, one scFv, named C6, which can specifically recognize CD99 on the surface of tumor cells, was obtained from a large CD99 phage antibody library prepared using the extracellular domain of CD99 as antigen. Sequencing analysis reveals that the nucleotide sequence of C6 is shown as SEQ ID NO. 2, and the amino acid sequence of C6 is shown as SEQ ID NO.1.

In order to characterize the advantages and disadvantages of the ScFv screened out in the present application relative to the ScFv against CD99 in the prior art and to determine whether it is suitable for the construction of chimeric antigen receptors, four ScFvs known in the prior art (C2 and C3 in CN 110590960 A; Sequences 47 and 83 in the WO 2019136419) were selected for further study. Next, four ScFvs known in the prior art are named C2, C3, C4 and C5, respectively, and their amino acid sequences are shown as SEQ ID NO.3, SEQ ID NO.5, SEQ ID NO.7 and SEQ ID NO.9, respectively, and their nucleotide sequences are represented by SEQ ID NO.4, SEQ ID NO.6, SEQ ID NO.8 and SEQ ID NO.10, respectively.

In order to determine the affinity of the above scFvs to the antigen CD99, the binding kinetics of the soluble scFv to the extracellular domain of CD99 was further analyzed by surface plasmon resonance analysis, and the Kd values of the purified C6, C2, C3, C4, and C5 were calculated as follows:

The nucleotide sequences shown in SEQ ID NO: 2, SEQ ID NO: 4, SEQ ID NO: 6, SEQ ID NO: 8 and SEQ ID NO: 10 were artificially synthesized with the cleavage site Nde I and Xho I each added to either terminal. The synthetic nucleotide sequences were subjected to dual-enzyme digestion, and then ligated to a cloning vector PMD-19T subjected to the same digestion process as above. The ligated product was transformed into the competent cells, and then the positive clones were screened and sequenced. The plasmid was extracted from the positive clone with a correct sequence, and then subjected to dual-enzyme digestion by using Nde I and Xho I. Meanwhile, an expression vector PET-28b was also subjected to the same dual-enzyme digestion as above. The nucleotide fragment was ligated with the vector fragment, with which E. coli was transformed. The positive clones were screened and sequenced. The positive clone with correct sequence was selected for extracting the plasmid. The scFv antibody was expressed in E. coli BL21 (DE3) transformed by the plasmid and purified, in order to obtain soluble C6, C2, C3, C4 and C5. BiacoreX was conducted to analyze the binding kinetics of the above scFvs to the extracellular domain of CD99 and to calculate their Kd values, which were 4.5x10-7 M, 4.8x10-7 M, 5.1x10-7 M, 6.0x10-7 M, 5.4x10-7 M for C6, C2, C3, C4, and C5, respectively.

The results showed that all 5 anti-CD99 scFvs studied in the present disclosure had higher affinity and could be used for the subsequent construction of CAR-T cells.

### Example 2: Construction of Plasmid PTK881-EF1α-C6, PTK881-EF1α-C2, PTK881-EF1α-C3, PTK881-EF1α-C4, PTK881-EF1α-C5, PTK881-EF1α-C6-7x19, PTK881-EF1α-C2-7x19, PTK881-EF1α-C3-7x19, PTK881-EF1α-C4-7x19, PTK881-EF1α-C5-7x19

1. Fragments C6, C2, C3, C4, and C5 were artificially synthesized, respectively, and fragments SP and strepII-CD8 hinge-CD28TM+ICD-4-1BB-CD3ζ were also artificially synthesized.

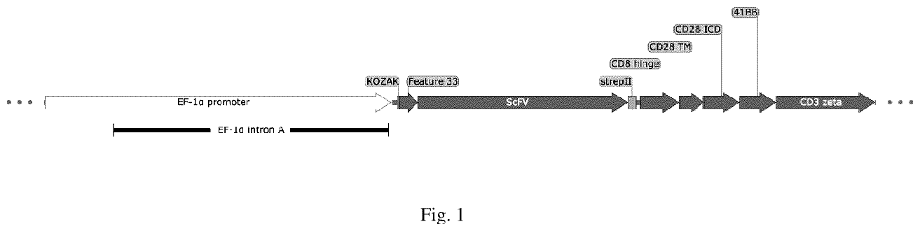

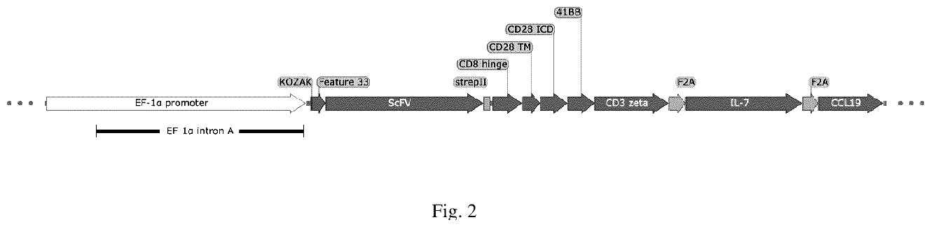

2. Using SP, C6/C2/C3/C4/C5 and strepII-CD8 hinge-CD28TM+ICD-4-1BB-CD3ζ, or using SP, C6/C2/C3/C4/C5 and strepII-CD8 hinge-CD28TM+ICD-4-1BB-CD3ζ+F2A peptide+IL-7+F2A peptide+CCL19 as templates, C6-CAR, C2-CAR, C3-CAR, C4-CAR, C5-CAR, 6-7x19 CAR, C2-7x19 CAR, C3-7x19 CAR, C4-7x19 CAR, and C5-7x19 CAR have been obtained with cleavage sites EcoR I and BamH I through amplification by Overlap PCR, wherein the structures of C6-CAR, C2-CAR, C3-CAR, C4-CAR, and C5-CAR are shown in FIG. 1, and the structures of C6-7x19 CAR, C2-7x19 CAR, C3-7x19 CAR, C4-7x19 CAR, and C5-7x19 CAR fragments are shown in FIG. 2.

Among others, the amino acid sequence of the signal peptide (SP) is shown as SEQ ID NO.11, the amino acid sequence of strepII is shown as SEQ ID NO.13, the amino acid sequence of the CD8 hinge is shown as SEQ ID NO.15, the amino acid sequence of CD28TM is shown as SEQ ID NO.17, The amino acid sequence of CD28ICD is shown as SEQ ID NO.19, the amino acid sequence of 4-1BB is shown as SEQ ID NO.21, the amino acid sequence of CD3ζ is shown as SEQ ID NO.23, the amino acid sequence of the F2A peptide is shown as SEQ ID NO.25, The amino acid sequence of IL-7 is shown as SEQ ID NO.27, the amino acid sequence of CCL19 is shown as SEQ ID NO.29, and more preferably, the nucleotide sequence of the signal peptide (SP) is shown as SEQ ID NO.12, the nucleotide sequence of strepII is shown as SEQ ID NO.14, The nucleotide sequence of the CD8 hinge is shown as SEQ ID NO.16, the nucleotide sequence of CD28TM is shown as SEQ ID NO.18, the nucleotide sequence of CD28ICD is shown as SEQ ID NO.20, the nucleotide sequence of 4-1BB is shown as SEQ ID NO.22, the nucleotide sequence of CD3ζ is shown as SEQ ID NO.24, the nucleotide sequence of the F2A peptide is shown as SEQ ID NO.26, the nucleotide sequence of IL-7 is shown as SEQ ID NO.28, and the nucleotide sequence of CCL19 is shown as SEQ ID NO.30.

3. The plasmid PTK881-EF1α-Kan was digested with restriction enzyme EcoR I and BamH I, and the products were electrophoresed on 0.8% agarose gel, harvested by cutting the bands from the gel and placed in Eppendorf tubes. Corresponding fragments were recovered with the agarose-gel recovery kit from Axygen. The purity and concentration of the products were determined. 4. The fragments were added into an Eppendorf tube in a molar ratio of 1:2, into which Exnase II ligase (Vazyme) and homologous recombinase 5×CEII buffer were added, and then the mixture was reacted at 37° C. for 0.5 h. 10 μl of the ligation solution was taken out, into which 100 μl of DH5a competent cell suspension was added, and then the mixture was kept in an ice bath for 30 min followed by heat shock at 42° C. for 90 s. After that, 500 μl of soc medium was added and the mixture was subsequently cultured at 37° C. and 220 rpm for 2 h. After 2 h, the Eppendorf tube was centrifuged at 4000×g for 1 min to remove 400 μl of excess liquid. The remaining liquid was spread on an LB plate and cultured at 37° C. for 12 h. A single colony was picked from the plate and inoculated into 5 ml of LB liquid medium and cultured at 37° C. and 220 rpm for 12 h.

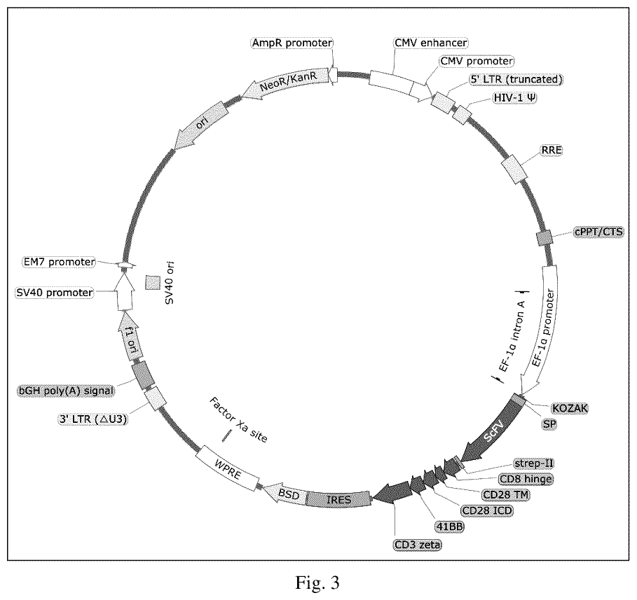

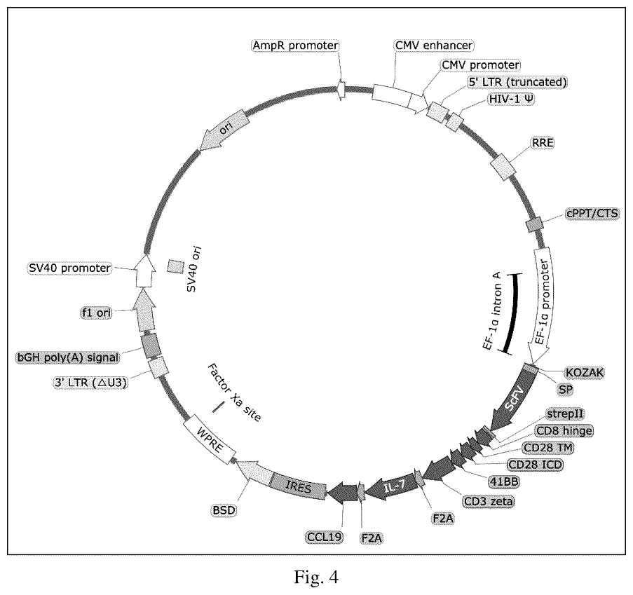

5. Plasmid PTK881-EF1α-C6, PTK881-EF1α-C2, PTK881-EF1α-C3, PTK881-EF1α-C4, PTK881-EF1α-C5, PTK881-EF1α-C6-7x19, PTK881-EF1α-C2-7x19, PTK881-EF1α-C3-7x19, PTK881-EF1α-C4-7x19, and PTK881-EF1α-C5-7x19 were obtained by extraction using a mini extraction kit from Axygen. After verification by the first-generation sequencing in Sangon Biotech (Shanghai) Co., Ltd., E. coli DH5a strains containing plasmid PTK881-EF1α-C6, PTK881-EF1α-C2, PTK881-EF1α-C3, PTK881-EF1α-C4, PTK881-EF1α-C5, PTK881-EF1α-C6-7x19, PTK881-EF1α-C2-7x19, PTK881-EF1α-C3-7x19, PTK881-EF1α-C4-7x19, and PTK881-EF1α-C5-7x19 were preserved. The complete maps of PTK881-EF1α-C6, PTK881-EF1α-C2, PTK881-EF1α-C3, PTK881-EF1α-C4, and PTK881-EF1α-C5 were schematically shown in FIG. 3, and the complete maps of PTK881-EF1α-C6-7x19, PTK881-EF1α-C2-7x19, PTK881-EF1α-C3-7x19, PTK881-EF1α-C4-7x19, and PTK881-EF1α-C5-7x19 were schematically shown in FIG. 4.

### Example 3. Preparation and Sequencing of the Plasmids

### 1. Preparation of the Plasmids

E. coli DH5a strains containing plasmid PTK881-EF1α-C6, PTK881-EF1α-C2, PTK881-EF1α-C3, PTK881-EF1α-C4, PTK881-EF1α-C5, PTK881-EF1α-C6-7x19, PTK881-EF1α-C2-7x19, PTK881-EF1α-C3-7x19, PTK881-EF1α-C4-7x19, PTK881-EF1α-C5-7x19 were inoculated into 250 ml of LB medium containing 100 μg/ml ampicillin, respectively, and cultured at 37° C. and 220 rpm overnight. The culture medium was centrifuged at 6 000×g for 20 min at 4° C., and the supernatant was discarded.

120 ml of pre-cooled Buffers P1 from the EndoFree plasmid mega kit (Qiagen) was added to the pellet of E. coli after centrifugation. The centrifuge bottle was covered and shaken vigorously to make the pellet completely dispersed in Buffers P1.

120 ml of Buffers P2 was added to a centrifuge bottle, which was then covered and placed on a roller mixer. The speed of the roller mixer was slowly increased to 50 rpm. After thoroughly mixing, the mixture was placed at room temperature for 5 min.

120 ml of Buffers P3 was added to a centrifuge bottle, which was then covered and placed on a roller mixer. The speed of the roller mixer was slowly increased to the maximum speed of 70 rpm. The mixture was thoroughly mixed until it became a white, non-viscous and fluffy mixture. The mixture was then centrifuged at 9000×g for 15 min at 4° C.

50 ml of Buffer FW was poured into QIAfilter Cartridge, and the supernatant obtained by centrifugation was also poured into QIAfilter Cartridge, and they were mixed gently. The mixture was suction-filtered into a corresponding glass bottle with a suitable label. 20 ml Buffer ER was added to each glass bottle, mixed upside-down for 6 times, and incubated at −20° C. for 30 min.

The labeled mega column was placed on a corresponding rack, and each mega column was equilibrated with 35 ml Buffers QBT, and drained out by gravity.

All of the liquid in the glass bottle was poured into a corresponding labeled mega column in batches. After the liquid was drained out, each mega column was rinsed by 200 ml Buffer QC added in batches. After the liquid in the column was drained out, the waste liquid in the waste liquid collection tray was poured into a 50 ml clean centrifuge tube.

40 ml Buffer QN was again added to each mega column, and the effluent was collected into a 50 ml clean centrifuge tube, mixed upside-down for 6 times. 20 ml of the effluent was then transferred to another 50 ml clean and labeled centrifuge tube.

14 ml of isopropanol (at room temperature) was added to each 50 ml centrifuge tube, and mixed upside-down for 6 times. The mixture was then centrifuged at 15000×g for 50 min at 4° C.

The supernatant was sucked out in a superclean bench. Each tube was carefully rinsed by 3.5 ml Endotoxin-free water to avoid breaking up the precipitate at the bottom. The mixture was then centrifuged at 15000×g for 30 min at 4° C. The Buffer TE from the EndoFree plasmid mega kit was preheated in an oven.

The supernatant obtained after centrifugation was sucked out and air-dried in a superclean bench (the residual absolute ethanol was removed by volatilization for about 10 min).

The Buffer TE was taken out of the oven. 1 ml Buffer TE was added to each tube in a superclean bench, and mixed by repeated pipetting for 10 times. Subsequently, the tube was place into an oven at 65° C. During this period, the tube wall was continuously knocked to make the precipitate dissolved completely. The mixture was centrifuged at 4000×g for 1 min at 4° C. The tube was shaken to make the liquid on the tube wall fall to the bottom. The mixture was mixed by repeated pipetting.

In a superclean bench, all the liquid was transferred to a corresponding labeled EP tube with no endotoxin, no pyrogen and no nuclease. 2 μl liquid was sucked out to determine the concentration of the plasmid using a micro-spectrophotometer. The concentration was labled on corresponding EP tube, and the plasmid PTK881-EF1α-C6, PTK881-EF1α-C2, PTK881-EF1α-C3, PTK881-EF1α-C4, PTK881-EF1α-C5, PTK881-EF1α-C6-7x19, PTK881-EF1α-C2-7x19, PTK881-EF1α-C3-7x19, PTK881-EF1α-C4-7x19, and PTK881-EF1α-C5-7x19 were obtained.

### 2. Target Gene Sequencing

20 μl (500 ng) plasmid DNA was taken out for sequencing. According to the original seed sequence, the product obtained from the plasmid preparation was checked to verify whether the target gene has changed. Under a stable process, the target gene will not change during fermentation scale-up of the working seeds, and can be used for the next step in the production process to express proteins correctly.

### Example 4. Preparation and Viable Titre Assay of the Lentiviral Vector PTK881-EF1α-C6, PTK881-EF1α-C2, PTK881-EF1α-C3, PTK881-EF1α-C4, PTK881-EF1α-C5, PTK881-EF1α-C6-7x19, PTK881-EF1α-C2-7x19, PTK881-EF1α-C3-7x19, PTK881-EF1α-C4-7x19, and PTK881-EF1α-C5-7x19

### 1. Preparation of the Lentiviral Vector

293T cells (Takara) were inoculated at a number of 130.0 to 140.0×10⁶ cells into a multilayer cell culture bottle (Hyperflask) containing a total of 560 ml of complete DMEM medium (50 ml of fetal bovine serum, 5 ml of Antibiotic-Antimycotic (100×)), and incubated at 37° C. in a incubator containing 5% CO₂ for 24 h. The complete DMEM medium mixed with 320 μg of the following plasmids (PTK881-EF1α-C6/PTK881-EF1α-C2/PTK881-EF1α-C3/PTK881-EF1α-C4/PTK881-EF1α-C5/PTK881-EF1α-C6-7x19/PTK881-EF1α-C2-7x19/PTK881-EF1α-C3-7x19/PTK881-EF1α-C4-7x19/PTK881-EF1α-C5-7x19:plasmid BZ1:plasmid BZ2:plasmid BZ3=12:10:5:6) was added to a 960 μg PEI tube, vortexed, and equilibrated at room temperature for 10 min. 35 ml of the mixture of PEI and the plasmids was then mixed with 525 ml of complete DMEM medium, and then transferred into a multilayer cell culture bottle. The multilayer cell culture bottle was placed in an incubator containing 5% CO₂ at 37° C. for 3 days, and then the supernatant from the cell culture was collected.

The supernatant was centrifuged at 4000 rpm (or 3000×g) for 30 min, and then cryonase (Takara) was added and kept at 4° C. After 6 hours, the lentivirus supernatant was suction-filtered using a 0.22 m filter and centrifuged at 4° C. for 2.5 h at 30000×g. The supernatant was removed, and 1 ml of the T-cell culture medium was added to re-suspend the pellet. After re-suspension, 20 μl was collected to determine the viable titer of virus, and the remaining lentivirus concentrate was aliquoted and labled as Lenti3-C6-CAR, Lenti3-C2-CAR, Lenti3-C3-CAR, Lenti3-C4-CAR, Lenti3-C5-CAR, Lenti3-C6-7x19-CAR, Lenti3-C2-7x19-CAR, Lenti3-C3-7x19-CAR, Lenti3-C4-7x19-CAR, and Lenti3-C5-7x19-CAR, and was stored at −80° C. for later use.

### 2. Detection of Viable Titre of Lentiviral Vector

Principle: anti-strepII antibody was labeled with fluorescein, and also could specifically bind to strepII in CAR. Thus, the expression of CAR in 293T cells can be indirectly reflected by the fluorescent signal detected by a flow cytometer.

Methods: 293T cells were inoculated at 5.0x10⁵ cells/well into a 6-well plate, and 0.1 μl, 0.5 μl, and 1 μl of lentivirus concentrate were added into each well, respectively. A negative control was set. The plate was incubated in an incubator containing 5% CO₂ at 37° C. Three days later, 293T cells were collected with Versene solution (Gibco) and subjected to flow cytometry to detect the proportion of CAR-positive 293T cells, which was then transformed to the viable titre of the lentivirus concentrate of Lenti3-C6-CAR, Lenti3-C2-CAR, Lenti3-C3-CAR, Lenti3-C4-CAR, Lenti3-C5-CAR, Lenti3-C6-7x19-CAR, Lenti3-C2-7x19-CAR, Lenti3-C3-7x19-CAR, Lenti3-C4-7x19-CAR, and Lenti3-C5-7x19-CAR.

At present, the viable titre of the lentivirus concentrate is in the range of 1×10⁸˜10×10⁸ (TU/ml), and the detection and analysis results are shown in Table 1. The results indicate that each lentiviral vector can obtain higher viable titer and can be used for subsequent preparation of chimeric antigen receptor immune cells.

TABLE 1
Analysis results of the viable titre of lentivirus

| Sample number | Viable titer (TU/ml) |
|---|---|
| Lenti3-C6-CAR | 2.7 × 10⁸ |
| Lenti3-C2-CAR | 2.8 × 10⁸ |
| Lenti3-C3-CAR | 2.5 × 10⁸ |
| Lenti3-C4-CAR | 2.3 × 10⁸ |
| Lenti3-C5-CAR | 2.1 × 10⁸ |
| Lenti3-C6-7x19-CAR | 2.2 × 10⁸ |
| Lenti3-C2-7x19-CAR | 2.0 × 10⁸ |
| Lenti3-C3-7x19-CAR | 2.1 × 10⁸ |
| Lenti3-C4-7x19-CAR | 2.2 × 10⁸ |
| Lenti3-C5-7x19-CAR | 2.0 × 10⁸ |

### Example 5. Detection of the Transduction Efficiency and Viability of C3 CAR-T Transfected at Different Time Points

C3 CART-T was used as an example to study the transduction efficiency and viability of CAR-T, in order to determine the appropriate time point for preparation of CAR-T cell by transfection of T-cell.

### 1. Preparation of CAR-T Cell Preparation:

100 ml peripheral blood was collected from a healthy donor, and mononuclear cells were isolated by Ficoll lymphocyte separation medium. After counting, CD3-positive cells were sorted using an appropriate amount of CD3 MicroBeads, human (Miltenyi), and cultured at a density of 1.0 to 2.0×10⁶ cells/ml in complete T-cell culture medium (OpTmizer™ CTS™ T-Cell Expansion Basal Medium, OpTmizer™ CTS T-Cell Expansion Supplement (Invitrogen), 500 IU/ml IL-2 (Shuanglu Pharmaceutical)). At the same time, 25 l of Dynabeads Human T-Activator CD3/CD28 (Invitrogen) was added per 10⁶ cells to activate T-cells.

After 24 h (Day1), 48 h (Day2) and 72 h (Day3), Lenti3-C6-CAR lentivirus vector was added at MOI=1 for transduction, mixed and incubated in a CO₂ incubator, and supplemented with an appropriate amount of the complete T-cell medium for further culture after 4 h.

After 24 hours of lentivirus transduction, the transduced C6 CAR-T cells were exchanged into the fresh complete T-cell medium. The density of viable cells was adjusted to 1.0-2.0×10⁶/ml, and the cells were continued to culture and expand for 10 to 20 days. The culture was observed and counted every day, and expanded by supplementing medium according to the number of cells. The cell density was constantly maintained at 1.0 to 2.0×10⁶/ml.

2. Detection of Transduction Efficiency and Viability of C6 CAR-T Cells 1.0×10⁶ T-cells and C3 CAR-T cells were each incubated with 1 μg/ml FITC-Protein-L at room temperature for 30 minutes. The cells were washed twice with normal saline, and then re-suspended in 100 μl PBS. Subsequently, 5 μl/test of 7AAD antibody was added and incubated at room temperature in dark for 10 min. Then, FITC and 7AAD fluorescence signals were detected by a flow cytometer to determine the ratio of FITC-positive cells and the ratio of 7AAD-negative cells, which reflected the ratio of CAR-T cells in total cells and the cell viability, respectively.

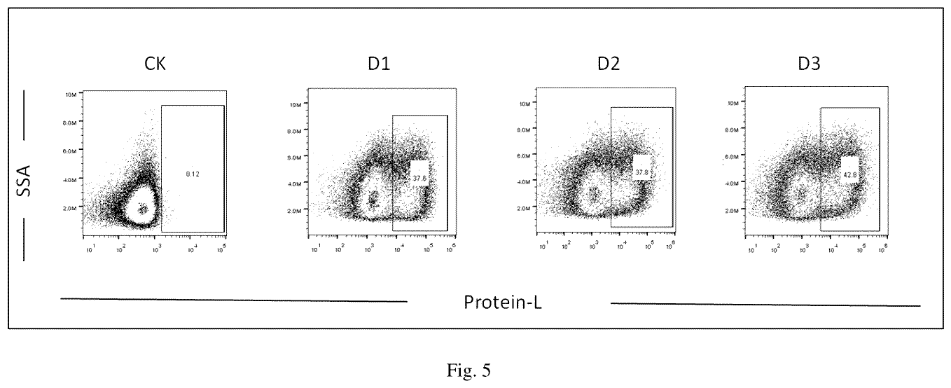

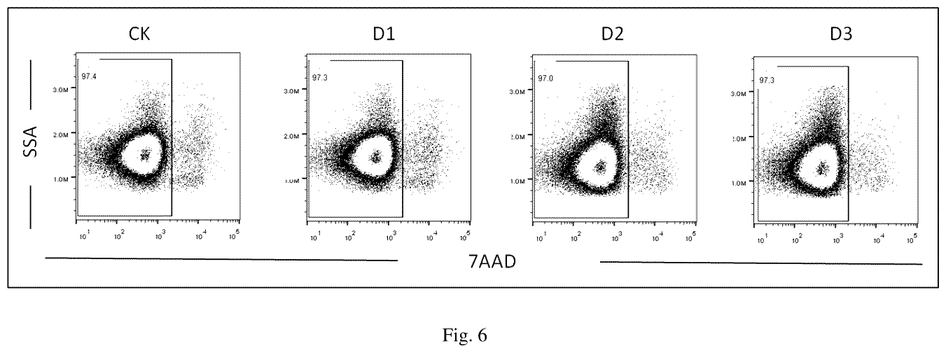

As shown in FIG. 5 and FIG. 6, at 24 h (Day1), 48 h (Day2), and 72 h (Day3), the transfection efficiency was 37.6%, 37.8%, and 42.8%, respectively, and the viability was 97.3%, 97.0%, and 97.3%, respectively. In order to ensure the optimal transfection efficiency and viability, CAR-T cells were prepared by transfecting T-cells at Day2, i.e., 48 h.

### Example 6. Preparation of C6 CAR-T, C2 CAR-T, C3 CAR-T, C4 CAR-T, C5 CAR-T, C6-7x19 CAR-T, C2-7x19 CAR-T, C3-7x19 CAR-T, C4-7x19 CAR-T, C5-7x19 CAR-T cells

### 1. Preparation of CAR-T Cell Preparation:

100 ml peripheral blood was collected from a healthy donor, and mononuclear cells were isolated by Ficoll lymphocyte separation medium. After counting, CD3-positive cells were sorted using an appropriate amount of CD3 MicroBeads, human (Miltenyi), and cultured at a density of 1.0 to 2.0×10⁶ cells/ml in complete T-cell culture medium (OpTmizer™ CTS™ T-Cell Expansion Basal Medium, OpTmizer™ CTS T-Cell Expansion Supplement (Invitrogen), 500 IU/ml IL-2 (Shuanglu Pharmaceutical)). At the same time, 25 l of Dynabeads Human T-Activator CD3/CD28 (Invitrogen) was added per 10⁶ cells to activate T-cells.

After 48 h (Day2), the lentivirus vector Lenti3-C6-CAR, Lenti3-C2-CAR, Lenti3-C3-CAR, Lenti3-C4-CAR, Lenti3-C5-CAR, Lenti3-C6-7x19-CAR, Lenti3-C2-7x19-CAR, Lenti3-C3-7x19-CAR, Lenti3-C4-7x19-CAR, and Lenti3-C5-7x19-CAR were added at MOI of 3 for transduction, mixed and incubated in a CO₂ incubator, and supplemented with an appropriate amount of the complete T-cell medium for further culture after 4 h.

After 24 hours of lentivirus transduction, the transduced cells were exchanged into the fresh complete T-cell medium. The density of viable cells was adjusted to 1.0-2.0×10⁶/ml, and the cells were continued to culture and expand for 10 to 20 days. The culture was observed and counted every day, and expanded by supplementing medium according to the number of cells. The cell density was constantly maintained at 1.0 to 2.0×10⁶/ml. 2. Detection of the transduction efficiency of C6 CAR-T, C2 CAR-T, C3 CAR-T, C4 CAR-T, C5 CAR-T, C6-7x19 CAR-T, C2-7x19 CAR-T, C3-7x19 CAR-T, C4-7x19 CAR-T, C5-7x19 CAR-T cells 1.0×10⁶ transduced T-cells were incubated with 1 μg/ml FITC-Protein-L at room temperature for 30 minutes, and washed twice with normal saline. The fluorescent signal of FITC was detected by a flow cytometer to determine the ratio of FITC-positive cells, which reflected the ratio of the CAR-T cells in total cells. The transduction efficiency of C6 CAR-T, C2 CAR-T, C3 CAR-T, C4 CAR-T, C5 CAR-T, C6-7x19 CAR-T, C2-7x19 CAR-T, C3-7x19 CAR-T, C4-7x19 CAR-T, and C5-7x19 CAR-T cells were listed in table 2. Table 2 shows that the CAR-T cells were successfully prepared, but the expression efficiency of CAR in C4 CAR-T and C5 CAR-T cells, and C4-7x19 CAR-T and C5-7x19 CAR-T cells was the lowest, only 36.3% and 42.0%, and 36.1% and 35.5%, respectively. They were significantly lower than the expression efficiencies in C6, C2, and C3-related CAR-T cells.

TABLE 2
Transduction efficiency of the CAR-T cells

| No. | Transduction type | Transduction efficiency |
|---|---|---|
| 1 | C6 CAR-T | 64% |
| 2 | C2 CAR-T | 58.8% |
| 3 | C3 CAR-T | 55.9% |
| 4 | C4 CAR-T | 36.3% |
| 5 | C5 CAR-T | 42.0% |
| 6 | C6-7x19 CAR-T | 50.3% |
| 7 | C2-7x19 CAR-T | 48.2% |
| 8 | C3-7x19 CAR-T | 55.9% |
| 9 | C4-7x19 CAR-T | 33.1% |
| 10 | C5-7x19 CAR-T | 39.5% |

### Example 7. Proliferative Capacity and In Vitro Function Assay of C6 CAR-T, C2 CAR-T, C3 CAR-T, C4 CAR-T, C5 CAR-T, C6-7x19 CAR-T, C2-7x19 CAR-T, C3-7x19 CAR-T, C4-7x19 CAR-T, C5-7x19 CAR-T cells

### 1. Cell Proliferation Capacity Assay

When CAR-T cells are used in scientific research or therapy, the proliferative capacity of cells is a very important indicator. Only when cells have good proliferative capacity can they obtain enough amount of CAR-T cells.

PKH26 is a red fluorescent dye for lipid staining in cells, which has good binding ability with cells, strong fluorescence and is not easy to quench. PKH26 is widely used for cell labeling and tracking. After division and proliferation of T-cells labeled by PKH26, the fluorescence intensity is weakened by a half after proliferation to the next generation, so the fluorescence intensity is significantly lower than that of the control T-cells not stimulated for division. Flow cytometry can be used for analysis in the detection channel FL2.

The analysis steps are briefly described as follows: 100 ml peripheral blood was collected from a healthy donor, and mononuclear cells were isolated by Ficoll lymphocyte separation medium. After counting, CD3-positive cells were sorted using an appropriate amount of CD3 MicroBeads, human, and cultured at a density of 1.0×10⁶ cells/ml in complete T-cell culture medium (OpTmizer™ CTS™ T-Cell Expansion Basal Medium, OpTmizer™ CTS T-Cell Expansion Supplement (Invitrogen), 500 IU/ml IL-2 (Shuanglu Pharmaceutical)). At the same time, 25 μl of Dynabeads Human T-Activator CD3/CD28 (Invitrogen) was added per 10⁶ cells to activate T-cells.

After 48 h of activation, the lentiviral vector Lenti3-C6-CAR, Lenti3-C2-CAR, Lenti3-C3-CAR, Lenti3-C4-CAR and Lenti3-C5-CAR, and Lenti3-C6-7x19-CAR, Lenti3-C2-7x19-CAR, Lenti3-C3-7x19-CAR, Lenti3-C4-7x19-CAR, and Lenti3-C5-7x19-CAR were added at MOI=1 for transduction, Mixed and incubated in a CO₂ incubator. After 4 h, an appropriate amount of complete T-cell medium was supplemented for culture.

1×10⁶ cells were taken out, into which serum-free medium was added for washing. The mixture was then centrifuged at 1500 rpm for 5 min, and supernatant was discarded. 1 ml of diluent C was added and pipetted gently to prepare cell suspension.

3 μl of PHK426 solution was added into 1 ml of diluent C, mixed well to prepare a staining solution. The cell suspension was quickly added into the staining solution, mixed immediately, and incubated in a incubator at 37° C. for 5 minutes in dark. The suspension was shaken every 2 minutes.

2 ml of serum was added and left to stand for 1 min to stop the reaction. The cells were mixed with 5 ml of the complete cell medium, centrifuged at 1200 rpm for 6 min, washed for 3 times, and re-suspended in the complete cell medium. In addition, 1× untransfected T-cells were labeled as the parent cells by PKH26, fixed with 4% paraformaldehyde and stored at 4° C. in dark for later use.

Fluorescently labeled T-cells were incubated with the purified recombinant extracellular domain of CD99 (final concentration of 5 μg/ml) for activation. Each group was set up with three duplicate wells, which were mixed and cultured in an incubator for 10 days. Unlabeled but stained T-lymphocytes were used as blank control.

The cells were collected and washed with PBS once, and the fluorescence intensity of the cells was detected by a flow cytometer.

Cell proliferation results are shown in Table 3.

TABLE 3
Results of CAR-T cell activation and proliferation

| Culture | | | | | | Fold of cell proliferation | | | | | |
|---|---|---|---|---|---|---|---|---|---|---|---|
| time (days) | C6 | C2 | C3 | C4 | C5 | C6-7 × 19 | C2-7 × 19 | C3-7 × 19 | C4-7 × 19 | C5-7 × 19 | T-cell |
| 0 | 1 | 1 | 1 | 1 | 1 | 1 | 1 | 1 | 1 | 1 | 1 |
| 2 | 6 | 5 | 8 | 6 | 5 | 8 | 8 | 10 | 5 | 4 | 3 |
| 4 | 15 | 18 | 20 | 13 | 14 | 30 | 32 | 40 | 18 | 16 | 10 |
| 6 | 30 | 30 | 32 | 22 | 20 | 62 | 40 | 56 | 26 | 25 | 15 |
| 8 | 55 | 48 | 60 | 42 | 40 | 95 | 92 | 122 | 44 | 46 | 30 |
| 10 | 104 | 112 | 120 | 78 | 80 | 180 | 156 | 180 | 82 | 80 | 56 |

The results showed that the proliferation capacity of the T-cells transfected with the chimeric antigen receptors was significantly improved after receiving activation signals, as compared with the untransfected T-cells. Unexpectedly, the proliferation capacity of C4-CAR-T and C5-CAR-T were significantly lower than that of C6-CAR-T, C2-CAR-T and C3-CAR-T. The addition of IL-7+CCL19 after the CD3ζ chain of the chimeric antigen receptor significantly improved the proliferation capacity of C6-CAR-T, C2-CAR-T and C3-CAR-T, especially C6-CAR-T. However, the addition of IL-7+CCL19 after the CD3ζ chain did not effectively enhance the proliferation capacity of C4-CAR-T and C5-CAR-T cells. Without being bounded by theory, the proliferation capacities of the CAR-T cells were significantly different due to the different properties of the selected anti-CD99 scFvs.

Therefore, Based on the requirement of CAR-T cell therapy for the CAR-T cell, the CAR-T cells constructed by the C6-, C2- and C3-containing scFv are preferably adopted for subsequent applications.

### 2. In Vitro Tumor Killing Test:

In vitro tumor killing test was conducted on T, C6 CAR-T, C2 CAR-T, C3 CAR-T, C4 CAR-T, C5 CAR-T, C6-7x19 CAR-T, C2-7x19 CAR-T, C3-7x19 CAR-T, C4-7x19 CAR-T, and C5-7x19 CAR-T cells using calcein assay.

The target cells were selected from the cell lines of Ewing's sarcoma (EWS), acute lymphoblastic lymphoma/leukemia (T-all), acute myeloid leukemia (AML), malignant glioma and breast cancer. The screening criteria were high or moderate expression of the CD99 target outside the membrane. The selected cell lines were shown in Table 4. The negative target cells in the experimental group were K562 and Raji (CD99-negative cell lines).

TABLE 4
Selection of the target cell lines for anti-CD99 CAR-T cells

| Tumor type | Cell lines |
|---|---|
| Ewing's sarcoma (EWS) | TC71, 6647 |
| Acute lymphoblastic lymphoma (T-all) | JURKAT, MOLT-4 |
| Acute myeloid leukemia (AML) | MOLM-13 |
| Malignant glioma | U373-MG, U251-MG |
| Breast cancer | MCF-7 |

An appropriate amount of the target cells were taken out. Calcein-acetohydroxymethyl ester (Calcein-AM) was added to a cell suspension (PBS, 5% fetal bovine serum) at a density of 1×10⁶/ml to a final concentration of 25 μM, and the mixture was incubated in an incubator for 30 min. After washing twice at room temperature, the cells were re-suspended to 0.5×10⁵ cells/ml. The cells were added to each wells of a 96-well plate at a density of 0.5×10⁵/ml. T, C6 CAR-T, C2 CAR-T, C3 CAR-T, C4 CAR-T, C5 CAR-T, C6-7x19 CAR-T, C2-7x19 CAR-T, C3-7x19 CAR-T, C4-7x19 CAR-T, and C5-7x19 CAR-T cells were added at multiplicity of infection of 25:1. The cells were incubated at 37° C. for 2-3 h. After incubation, the fluorescence intensity of calcein in the supernatant was measured, and the lysis percentage of target cell was calculated based on the spontaneous release control and the maximal release control.

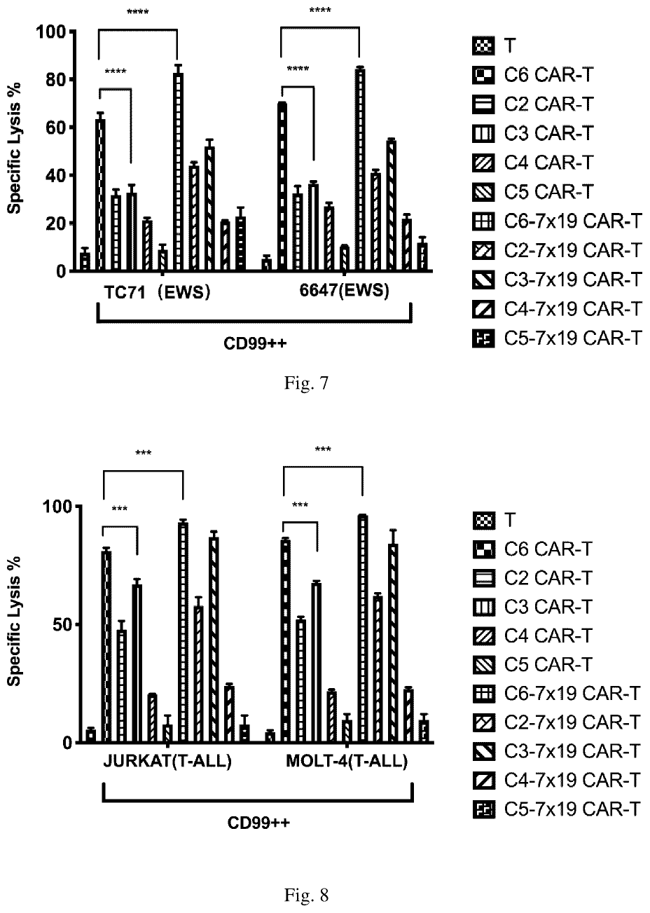

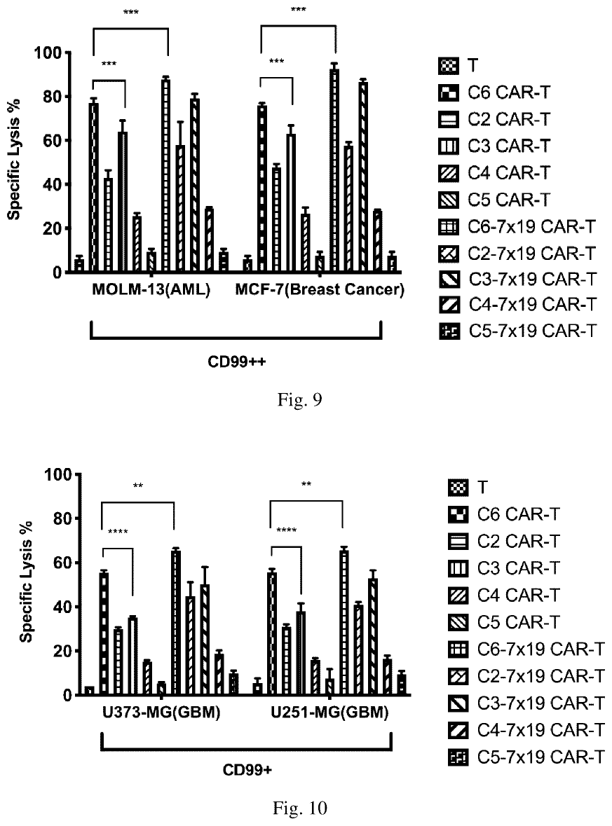

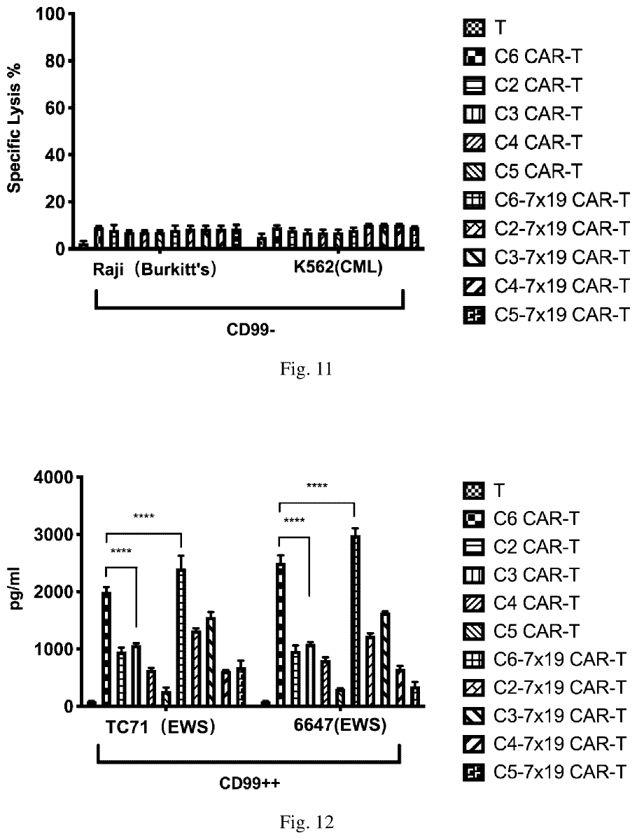

In vitro killing and lysis results of Ewing's sarcoma cell lines TC71 and 6647 with high expression of CD99, acute lymphoblastic lymphoma cell lines JURKAT and MOLT-4 with high expression of CD99, acute myeloid leukemia cell line MOLM-13 and breast cancer cell line MCF-7 with high expression of CD99, malignant glioma cell lines U373-MG and U251-MG with high expression of CD99, and K562 and Raji cell lines with no CD99 expression each by T, C6 CAR-T, C2 CAR-T, C3 CAR-T, C4 CAR-T, C5 CAR-T, C6-7x19 CAR-T, C2-7x19 CAR-T, C3-7x19 CAR-T, C4-7x19 CAR-T, and C5-7x19 CAR-T cells were shown in FIG. 7, FIG. 8, FIG. 9, FIG. 10, and FIG. 11, respectively.

The results indicated that as compared with T-cells, C6 CAR-T, C2 CAR-T, C3 CAR-T, C4 CAR-T, C5 CAR-T, C6-7x19 CAR-T, C2-7x19 CAR-T, C3-7x19 CAR-T, C4-7x19 CAR-T, and C5-7x19 CAR-T cells had higher targeted lysis capacity, demonstrating that it had significant in vitro killing function on Ewing's sarcoma, acute lymphoblastic lymphoma, acute myeloid leukemia, malignant glioma and breast cancer cell lines. Besides, in a co-incubation system with TC71, 6647, JURKAT, MOLT-4, MOLM-13, MCF-7, U373-MG, and U251-MG cell lines, the targeted lysis capacity of C6 CAR-T cells was significantly higher than that of C2 CAR-T, C3 CAR-T, C4 CAR-T and C5 CAR-T cells. It was also suggested by the results that the addition of IL-7+CCL19 after the CAR structure could significantly enhance the targeted lysis capacity of C6-7x19 CAR-T, C2-7x19 CAR-T and C3-7x19 CAR-T cells, but could not enhance the targeted lysis capacity of C4-7x19 CAR-T and C5-7x19 CAR-T cells. From the results above, it can be seen that C6 CAR-T has stronger lysis on Ewing's sarcoma, acute lymphoblastic lymphoma, acute myeloid leukemia, malignant glioma and breast cancer cell lines. The expression of IL-7+CCL19 in CAR-T cells can significantly enhance the tumor-killing capacity of C6 CAR-T, C2 CAR-T and C3 CAR-T. Without being bounded by theory, the difference in the cancer cell lysis capacities of the chimeric antigen receptors of different ScFvs may be resulted from their recognition of different CD99 epitopes and/or the difference in the properties of the ScFvs themselves.

As can be seen from the above in vitro tumor-killing results, C6 CAR-T, C2 CAR-T, and C3 CAR-T cells constructed from C6, C2, and C3 are preferred, and C6-7x19 CAR-T, C2-7x19 CAR-T, and C3-7x19 CAR-T are more preferred for the treatment of tumors.

### 3. Detection of Cytokine In Vitro:

An appropriate amount of the target cells were taken out. In a cell suspension (PBS, 5% fetal bovine serum) at a density of 1×10⁶/ml, after washing twice at room temperature, the cells were re-suspended to 0.5×10⁵/ml. The cells were added to each wells of a 96-well plate at a density of 0.05x10⁵ target cells/ml. T, C6 CAR-T, C2 CAR-T, C3 CAR-T, C4 CAR-T, C5 CAR-T, C6-7x19 CAR-T, C2-7x19 CAR-T, C3-7x19 CAR-T, C4-7x19 CAR-T, and C5-7x19 CAR-T cells were added at multiplicity of infection of 25:1. The mixture was centrifuged at 200×g for 30 seconds, and incubated at 37° C. for 18 h. After incubation, the concentration of IFN-γ in the supernatant was measured.

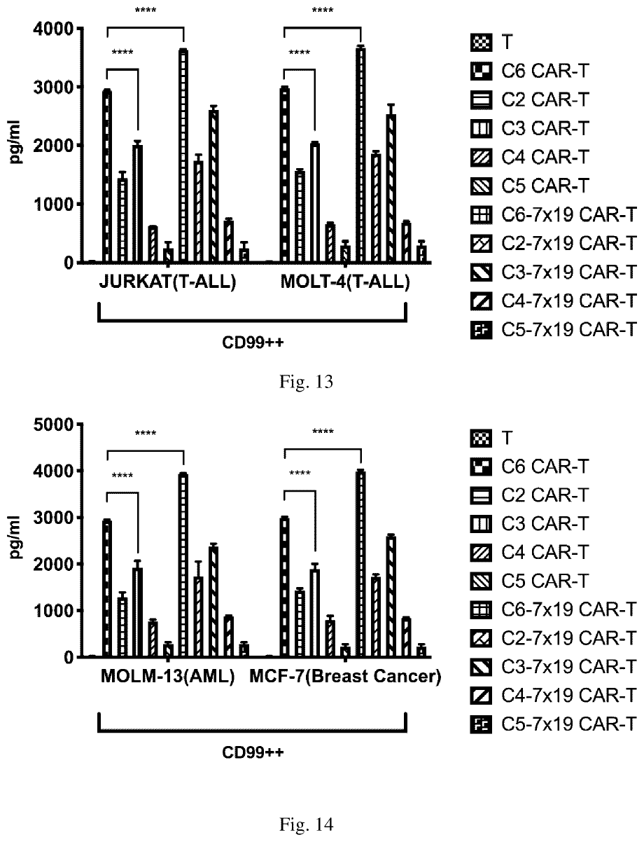

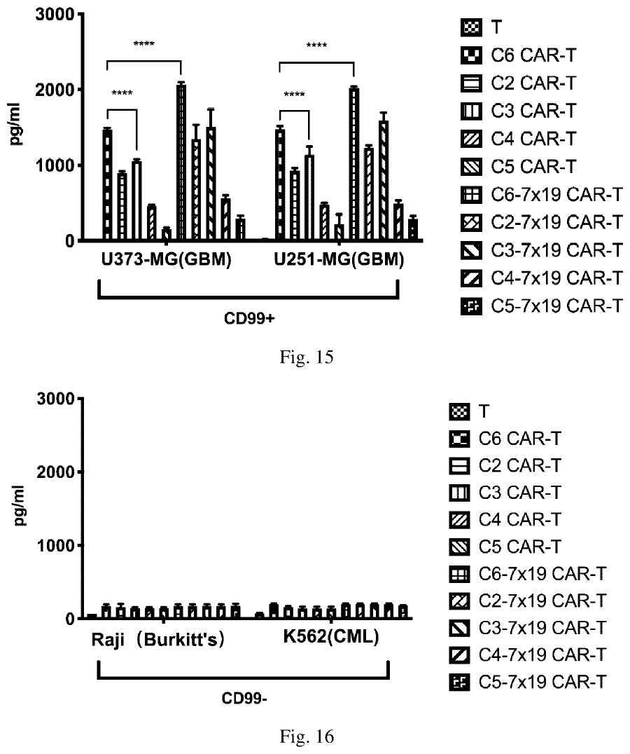

The results of IFN-γ secretion by T, C6 CAR-T, C2 CAR-T, C3 CAR-T, C4 CAR-T, C5 CAR-T, C6-7x19 CAR-T, C2-7x19 CAR-T, C3-7x19 CAR-T, C4-7x19 CAR-T, and C5-7x19 CAR-T cells after in vitro incubation with Ewing's sarcoma cell lines TC71 and 6647 with high expression of CD99, acute lymphoblastic lymphoma cell lines JURKAT and MOLT-4 with high expression of CD99, acute myeloid leukemia cell line MOLM-13 and breast cancer cell line MCF-7 with high expression of CD99, malignant glioma cell lines U373-MG and U251-MG with low expression of CD99, and K562 and Raji cell lines with no CD99 expression are shown in FIG. 12, FIG. 13, FIG. 14, FIG. 15, and FIG. 16, respectively. As indicated by the concentrations of IFN-γ, which is consistent with the results of killing tumor, the amounts of IFN-γ secreted by C6 CAR-T, C2 CAR-T, C3 CAR-T, C4 CAR-T, C5 CAR-T, C6-7x19 CAR-T, C2-7x19 CAR-T, C3-7x19 CAR-T, C4-7x19 CAR-T, and C5-7x19 CAR-T cells were all significantly enhanced as compared with T-cells, Which further demonstrated its in vitro killing effect on Ewing's sarcoma, acute lymphoblastic lymphoma, acute myeloid leukemia, malignant glioma and breast cancer cell lines. In a co-incubation system with TC71, 6647, JURKAT, MOLT-4, MOLM-13, U373-MG, U251-MG, MCF-7 cell lines, The release of IFN-γ from C6 CAR-T cells was significantly higher than that from C2-CAR-T, C3-CAR-T, C4 CAR-T and C5 CAR-T cells. It was also suggested by the results that the addition of IL-7+CCL19 after the CAR structure could significantly enhance the targeted lysis capacity of C6-7x19 CAR-T, C2-7x19 CAR-T and C3-7x19 CAR-T cells, but could not enhance the targeted lysis capacity of C4-7x19 CAR-T and C5-7x19 CAR-T cells. Likewise, without being bound by theory, the difference in the capacities of the chimeric antigen receptors of different ScFvs to stimulate the release of cytokines may be due to their recognition of different CD99 epitopes and/or the difference in the properties of the ScFvs themselves.

From the above results, it can be concluded that C6 CAR-T has stronger lysis capacity on Ewing's sarcoma, acute lymphoblastic lymphoma, acute myeloid leukemia, malignant glioma and breast cancer cell lines. The expression of IL-7+CCL19 in CAR-T cells can significantly enhance the tumor-killing capacity of C6 CAR-T, C2 CAR-T and C3 CAR-T.

Although embodiments of the present disclosure have been shown and described above, it is to be understood that the above-described embodiments are illustrative and not restrictive of the present disclosure, and that variations, modifications, substitutions, and alterations may be made thereto by those skilled in the art without departing from the scope of the present disclosure.

1. A chimeric antigen receptor, wherein the receptor comprises a signal peptide, a single-chain antibody ScFv, strepII, a CD8 hinge, a CD28 transmembrane region, a CD28 intracellular domain, an intracellular co-stimulatory domain 4-1BB, and a CD3ζ chain, wherein the amino acid sequence of the single-chain antibody ScFv is shown as SEQ ID NO. 1.

2. The chimeric antigen receptor of claim 1, wherein an F2A peptide, IL-7, an F2A peptide, and CCL19 are further spliced to the C-terminus of the CD3ζ chain in the receptor.

3. The chimeric antigen receptor of claim 2, wherein the amino acid sequence of the F2A peptide is represented by SEQ ID NO.25, the amino acid sequence of IL-7 is represented by SEQ ID NO.27, and the amino acid sequence of CCL19 is represented by SEQ ID NO.29.

4. The chimeric antigen receptor of claim 1, wherein the nucleotide sequence of the single-chain antibody ScFv is represented by SEQ ID NO.2.

5. A recombinant vector of the chimeric antigen receptor gene, wherein the vector is a lentiviral vector using the vector PTK881-EF1α as a backbone, and with the nucleotide sequence encoding the chimeric antigen receptor of claim 1 inserted.

6. A chimeric antigen receptor-expressing immune cell, wherein the immune cell is obtained by transfecting the immune cell with the nucleotide sequence encoding the chimeric antigen receptor of claim 1, and the immune cell is selected from T-cell, NK cell, NKT cell, αβT cell, γδT cell, CD4+ T-cell and CD8+ T-cell derived from umbilical cord blood, peripheral blood or iPSC.

7. Use of the chimeric antigen receptor of claim 1, the recombinant vector of the chimeric antigen receptor gene of claim 5, and the chimeric antigen receptor-expressing immune cell of claim 6, the preparation of drugs or kits for the treatment, prevention and diagnosis of tumors.

8. The use of claim 7, wherein the tumor is selected from the group consisting of Ewing's sarcoma, acute lymphoma/leukemia, acute myeloid leukemia, malignant glioma, and breast cancer.

9. The use of claim 8, wherein the tumor is acute T-cell lymphoid leukemia.

10. A method for preparing the chimeric antigen receptor-expressing immune cell of claim 6, wherein the method comprises the steps of: activating the isolated immune cell for 2-15 days, and infecting the immune cell with the recombinant vector of a chimeric antigen receptor gene to obtain the chimeric antigen receptor-expressing immune cell, wherein the immune cells are T-cells, wherein the recombinant vector of the chimeric antigen receptor gene, wherein the vector is a lentiviral vector using the vector PTK881-EF1α as a backbone, and with the nucleotide sequence encoding the chimeric antigen receptor inserted.

11. The recombinant vector of the chimeric antigen receptor gene of claim 5, wherein the nucleotide sequence of the single-chain antibody ScFv is represented by SEQ ID NO.2.

12. The recombinant vector of the chimeric antigen receptor gene of claim 11, wherein the amino acid sequence of the F2A peptide is represented by SEQ ID NO.25, the amino acid sequence of IL-7 is represented by SEQ ID NO.27, and the amino acid sequence of CCL19 is represented by SEQ ID NO.29.

13. The recombinant vector of the chimeric antigen receptor gene of claim 5, wherein the nucleotide sequence of the single-chain antibody ScFv is represented by SEQ ID NO.2.
